(19) United States
(12) Patent Application Publication
(10) Pub. No.: US 2008/0197106 A1
(43) Pub. Date: Aug. 21, 2008

(54) Method for manufacturing semiconical microneedles and semiconical microneedles manufacturable by this method

(76) Inventor: Ando Feyh, Tamm (DE)

Correspondence Address: KENYON & KENYON LLP, ONE BROADWAY, NEW YORK, NY, 10004, US

(21) Appl. No.: 12/011,711

(22) Filed: Jan. 28, 2008

(30) Foreign Application Priority Data
Jan. 29, 2007 (DE) 10 2007 004 344.0

(51) Int. Cl. C23F 17/00; A61M 5/00

(52) U.S. Cl. 216/2; 604/264

(57) ABSTRACT

A method for manufacturing semiconical microneedles in an Si-semiconductor substrate and a semiconical microneedles manufacturable made by this method.

### RELATED APPLICATION INFORMATION

The present application is based on priority German patent application no. 10 2007 004 344.0, which was filed in Germany on Jan. 29, 2007, and the disclosure of the foregoing German patent application is incorporated herein by reference.

### FIELD OF THE INVENTION

The present invention relates to a method for manufacturing semiconical microneedles in Si-semiconductor substrates and to semiconical microneedles manufacturable by this method.

### BACKGROUND INFORMATION

Microneedles as disposable products are subject to an increased cost pressure. One requirement for manufacturing microneedles is therefore the simplest possible, cost- and time-effective manufacturing process.

Methods for manufacturing microneedles are discussed, for example, in U.S. Pat. No. 6,334,856, which discusses the manufacturing of microneedles. Some known manufacturing methods form microneedles in a complicated process and in several process steps. In addition, the supply of fluids to the substrate and to the microneedle structure to be obtained must be regulated, which may have to take place both from the front and the back side of the substrate, depending on the process sequence.

Conical microneedles are normally manufactured by isotropic etching of a silicon semiconductor substrate. The conical microneedles manufacturable by isotropic etching have wide structures and are suitable as microneedles only conditionally if deeper penetration of the microneedles into the skin is required. In particular, if the edge of the needle tip is too wide, the needle tip may occasionally lose the sharpness needed for easily penetrating into the skin.

### SUMMARY OF THE INVENTION

The method according to the present invention for manufacturing semiconical microneedles in Si-semiconductor substrates has the advantage over the related art that microneedles having highly pointed and, at the same time, mechanically stable structures are manufacturable.

This is achieved according to the exemplary embodiments and/or exemplary methods of the present invention by the method having the following steps:
a) applying and structuring a first masking layer on the outer surface of the front of an Si-semiconductor substrate, discrete holes having straight lateral edges and an average diameter in the range of ≧50 μm to ≦1000 μm being formed in the first masking layer;
b) producing recesses having vertical lateral walls in the Si-semiconductor substrate by anisotropic etching into the discrete holes of the first masking layer of the Si-semiconductor substrate, the lateral walls of the produced recesses forming a vertical wall of the semiconical microneedles;
c) removing the first masking layer;
d) applying and structuring a second masking layer on the outer surface of the front of the Si-semiconductor substrate, the recesses remaining masked and adjacent areas along the lateral edges of the recesses being masked, these areas being covered in a semicircular shape;
e) isotropically etching the front of the Si-semiconductor substrate, during which the conical wall of the semiconical microneedles is formed;
f) optionally porosifying the front of the Si-semiconductor substrate;
g) removing the second masking layer;
h) optionally separating the semiconical microneedles from the Si-semiconductor substrate.

Furthermore, the method according to the present invention makes it possible to manufacture semiconical microneedles with the aid of a method permitting pure front side processing of an Si-semiconductor substrate.

The method according to the present invention is also advantageous in that it makes it possible to manufacture semiconical microneedles from a silicon semiconductor substrate cost-effectively because no complex fluid supplies through the substrate, for example, through a silicon wafer, are required.

In addition, the method according to the present invention makes it possible to manufacture an array of semiconical microneedles which may have a reservoir on the front for the substances to be injected, for example, active substances, in particular drugs. An array is a system of several, or a plurality of, microneedles on a support, which may be on an Si-semiconductor substrate.

The term “semiconical microneedle,” as defined herein, means a microneedle having a shaft in the form of a cone which has a vertical outer wall.

The exemplary embodiments and/or exemplary methods of the present invention is now described in greater detail with reference to FIGS. 1 through 5.

### BRIEF DESCRIPTION OF THE DRAWINGS

FIG. 1 shows a cross section of a semiconical microneedle manufacturable according to the present invention.

FIG. 2 shows a section through an Si-semiconductor substrate having a recess and an applied masking layer.

FIG. 3a shows a top view onto FIG. 2.

FIG. 3b shows a top view onto FIG. 2, a channel being provided in the vertical wall of the semiconical microneedles in an alternative specific embodiment.

FIG. 4 shows a section through an Si-semiconductor substrate having a semiconical microneedle structure according to the present invention adjacent to a central recess in the Si-semiconductor substrate.

FIG. 5 shows a section through an Si-semiconductor substrate having a semiconical microneedle structure according to the present invention adjacent to a central recess in the Si-semiconductor substrate, the semiconical microneedles and a layer of the Si-semiconductor substrate thereunder having been porosified.

### DETAILED DESCRIPTION

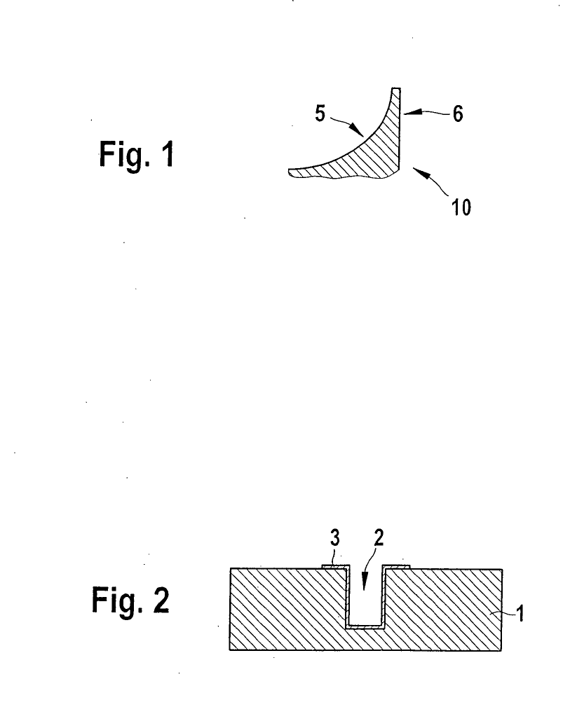

FIG. 1 shows a cross section of a semiconical microneedle structure 10 as manufacturable by the method according to the present invention. The semiconical microneedle has a conical outer wall 5 and a vertical outer wall 6.

FIG. 2 shows an Si-semiconductor substrate 1, in particular a silicon wafer which has a recess 2. For example, a photoresist layer having a thickness in the range of 1 μm to 2 μm has been applied to a p++ doped silicon wafer having a doping of 10¹⁹/cm³ as the first masking layer and structured, discrete square holes having an average diameter of 100 μm, for example, having been produced. Recess 2 has been produced by anisotropic etching, which may be via trenching through the holes. The depth of recess 2 is in the range of the later needle height or greater. For example, an etching depth of 100 μm has been produced at an etch rate of 7 μm/min in an etching time of 14 minutes. The first masking layer was subsequently removed. A second masking layer 3 was applied to Si-semiconductor substrate 1 having recess 2 and was structured. Masking layer 3 masks and passivates recess 2 in Si-semiconductor substrate 1. Masking layer 3 may be an SiN or Si₃N₄ layer, for example, having a thickness of approximately 150 nm.

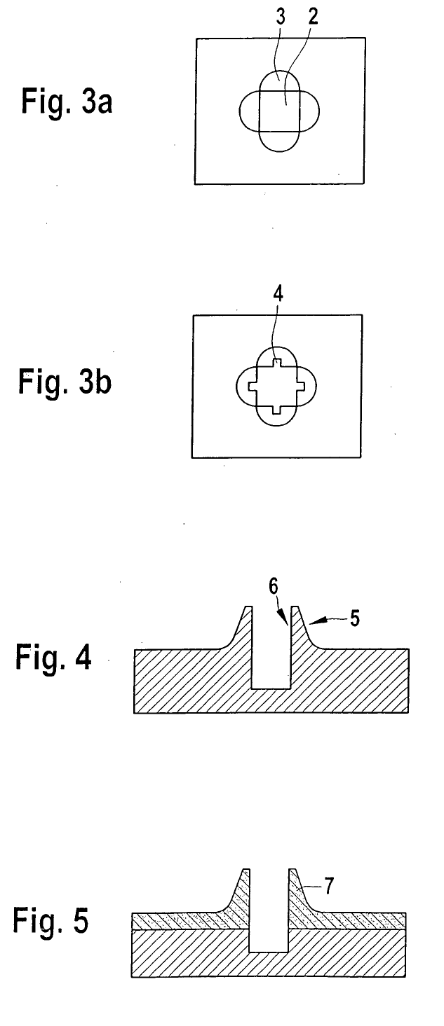

FIG. 3a shows a top view onto FIG. 2, masking layer 3 covering the areas of Si-semiconductor substrate 1 adjacent to the lateral walls of recess 2. In the areas adjacent to the lateral walls of recess 2, masking layer 3 has a semicircular structure. The tip of the later semiconical microneedle is formed in the areas which are masked by the semicircular structure.

In alternative specific embodiments such as shown in FIG. 3b, each channel 4 may be provided in the vertical outer wall in the semiconical microneedles, for example, via trenching. Each channel 4 is connected to central recess 2.

FIG. 4 shows a section through an Si-semiconductor substrate after isotropic etching. The semiconical microneedles according to the present invention surround a recess 2 in Si-semiconductor substrate 1 and have a conical outer wall 5 and a vertical outer wall 6. Conical outer wall 5 was produced by isotropic etching of the front of Si-semiconductor substrate 1, for example, in a 20% (vol/vol) aqueous hydrofluoric acid solution. An etching depth of 100 μm was achievable, for example, at a current density of 800 mA/cm² and an etching rate of approximately 16 μm/min in an etching time of 6 minutes.

FIG. 5 shows a section through an Si-semiconductor substrate 1 having a semiconical microneedle structure according to the present invention adjacent to a recess 2 in the Si-semiconductor substrate. The front of the Si-semiconductor substrate and the semiconical microneedle structures were porosified electrochemically using a hydrofluoric acid-containing etching medium. A 50% porosity was achieved, for example, using a 20% (vol/vol) aqueous hydrofluoric acid solution at a current density of 100 mA/cm². Accordingly, porosified semiconical microneedles 7 were obtained. At an etch rate of 75 nm/s, for example, an etching time of 11 minutes was needed for an etching depth of 50 μm. The second masking layer was attacked and partially dissolved already during etching in the aqueous hydrofluoric acid solution. The masking layer was completely dissolved subsequently during a ten-minute storage of the Si-semiconductor substrate in the electrolyte.

Exemplary methods according to the present invention are described herein and are elucidated in greater detail below.

Discrete through holes are formed in the first masking layer. The term “hole” as defined herein means an area of the masking layer which has a through opening in the masking layer, exposing the outer surface of the Si-semiconductor substrate. The holes allow access of the etching medium to the Si-semiconductor substrate. As defined herein, the term “discrete” means that the individual holes are not connected to each other. The holes may be evenly spaced.

The holes in the masking layer may have a polygonal shape. The polygon may be equilateral or may have lateral edges of different lengths. The polygons may be equilateral. The polygon may have a triangular or quadrangular, which may be a square, shape.

In certain exemplary embodiments, the average diameter of the holes of the first masking layer is in the range of ≧50 μm to ≦800 μm, which may be in the range of ≧75 μm to ≦500 μm, and may be in the range of ≧100 μm to ≦200 μm.

In certain exemplary embodiments, square-shaped recesses are formed. In further exemplary embodiments, rectangular recesses are formed.

One advantage of the square-shaped recesses is that a uniform system of recesses and microneedles on the Si-semiconductor substrate is made possible.

The depth of the recesses is in the range of the later needle height, or the recesses are deeper. In certain exemplary embodiments, the depth of the recesses is in the range of ≧100 μm to ≦500 μm, which may be in the range of ≧150 μm to ≦250 μm.

A depth of the recesses in the range of the later needle height may provide the advantage that, when using a system of a plurality of microneedles on a support, which may be a system on a layer of the Si-semiconductor substrate, a good system stability may be provided.

A depth of the recesses that is greater than the height of the microneedles may provide the advantage that, when using the recesses as a reservoir for the active substances or drugs to be injected, a larger volume may be contained.

In certain exemplary embodiments, the average diameter of the recesses is in the range of ≧50 μm to ≦1000 μm, which may be in the range of ≧50 μm to ≦800 μm, and further may be in the range of ≧75 μm to ≦500 μm, and may be in the range of ≧100 μm to ≦200 μm. In further exemplary embodiments, an average diameter of the recesses in the range of ≧50 μm to ≦200 μm, which may be in the range of ≧100 μm to ≦150 μm, is produced.

P-doped silicon wafers are well-suited may be Si-semiconductor substrates. For example, commercially available silicon wafers may be used.

Recesses having vertical lateral walls are manufacturable by anisotropic etching of the Si-semiconductor substrate. Exemplary methods include dry etching methods, in particular so-called trench methods, for example, the method known as Plasma Reactive Ion Etching (Plasma RIE) or deep trench methods. The so-called Bosch process is particularly well-suited. Suitable methods are described, for example, in “Laermer et al., ‘Bosch Deep Silicon Etching: Improving Uniformity and Etch Rate for Advanced MEMS Applications,’ Micro Electro Mechanical Systems, Orlando, Fla., USA, (Jan. 17-21, 1999).”

For this purpose, a first masking layer is applied onto the Si-semiconductor substrate, i.e., the silicon wafer, which is exposed using a so-called trench mask and subsequently structured using photolithographic methods. SiN, Si₃N₄, or SiC layers are suitable as a masking layer, for example. The masking layer may also be formed from other substances, for example, a photoresist. The exposed and structured masking layer is known as “etching mask.” It is advantageous in particular that anisotropic etching of the front of the Si-semiconductor substrate, i.e., the silicon wafer, may take place.

The semiconical microneedle manufacturable by the method according to the present invention may be designed without a through opening in the microneedle or in the form of a hollow needle. The term “hollow needle” as defined herein means that the semiconical microneedle has a through opening, i.e., a through channel through the inside of the microneedle structure.

In advantageous specific embodiments, a channel may be formed in the semiconical microneedle by anisotropic etching of the Si-semiconductor substrate. A channel may be formed in or near the vertical wall of the semiconical microneedle. The vertical wall of a semiconical microneedle is formed by the lateral walls of the recesses produced by anisotropic etching of the Si-semiconductor substrate, which may be by the trench method. The channel may be connected to the recess. In certain exemplary embodiments, each channel of the semiconical microneedles surrounding a central recess is connected to the recess. The channel may have different cross-section shapes; the channel may have a round or quadrangular cross section.

A channel may be formed by selecting a suitable masking layer or etching mask by anisotropic etching of the Si-semiconductor substrate, which may be together with the anisotropic etching of the recesses. This provides the advantage that no further method step is needed. It may be provided that a channel in the semiconical microneedles is formed in a separate method step.

A channel in the structure of the semiconical microneedles may provide, for example, a transport channel for the supply of active substances or drugs.

The first masking layer is removed after the anisotropic etching. In further method steps, a second masking layer is applied to the Si-semiconductor substrate for the isotropic etching. The isotropic etching is performed from the front of the Si-semiconductor substrate, the conical walls of the semiconical microneedles being formed.

The recesses produced by anisotropic etching remain covered by the second masking layer. The recesses may be passivated by the masking layer. This passivation protects the recesses from further isotropic etching. Furthermore, the second masking layer masks the areas adjacent to the recesses along the lateral edges, these areas being covered in a semicircular shape. These areas covered in a semicircular shape are underetched laterally by the isotropic etching. The structures of the Si-semiconductor substrate remaining under the areas masked in a semicircular shape form the tips of the semiconical microneedles.

The isotropic etching of the front of the Si-semiconductor substrate, during which a conical wall of a semiconical microneedle is formed, which may take place by electrochemical anodizing, and which may be in a hydrofluoric acid-containing electrolyte. Furthermore, dry etching methods using gases which etch silicon isotropically, which may be selected from the group including SF₆, XeF₂ and/or ClF₃, may be used.

The Si-semiconductor substrate, for example, a silicon wafer, may be used as the anode in anodic electrochemical etching processes. Isotropic etching may be performed in hydrofluoric acid-containing electrolytes, in particular in aqueous hydrofluoric acid solutions or mixtures containing hydrofluoric acid, water, and further solvents, for example, alcohols, in particular selected from the group including ethanol and/or isopropanol.

The process of the complete electrochemical dissolution of silicon is also known as electropolishing. Exemplary current densities for isotropic etching in aqueous hydrofluoric acid solutions may be in the range of ≧10 mA/cm² to ≦4000 mA/cm², and may also be in the range of between ≧50 mA/cm² to ≦500 mA/cm². Certain exemplary hydrofluoric acid concentrations are in the range of between ≧10% by volume to ≦40% by volume, in relation to the total volume of the etching solution. In certain exemplary embodiments, the etching rates may be in the range of ≧0.1 μm/s to ≦20 μm/s, and may be in the range of ≧1 μm/s to ≦10 μm/s.

Isotropic etching (electropolishing) of an Si-semiconductor substrate, for example, in hydrofluoric acid, which may have a lateral etching rate of 70% of the vertical etching rate.

In further specific embodiments of the method according to the present invention it may be provided that porosified semiconical microneedles are manufactured. The semiconical microneedles may be porosified by electrochemical anodizing. The porosifying process may be performed in a hydrofluoric acid-containing electrolyte.

One particular advantage of the method may be provided in particular exemplary embodiments by porosifying after isotropic etching via so-called electropolishing, for example, in hydrofluoric acid-containing electrolytes, by reducing the current density without having to change the etching medium in further method steps.

Certain exemplary current densities for porosifying the Si-semiconductor substrate are in the range of 10 mA/cm² to 400 mA/cm², and may be in the range of between 50 mA/cm² to 150 mA/cm².

The porosity of silicon is adjustable by suitable selection of the process parameters, for example, of the electrolyte composition, in particular of the hydrofluoric acid concentration, or of the current density.

The porosity of the semiconical microneedles may be in the range of ≧10% to ≦80%, and may be in the range of ≧25% to ≦60%. A porosity of the semiconical microneedles of less than 50% may advantageously provide an advantageous mechanical stability of the semiconical microneedles.

“Porosity” in the sense used herein is defined so as to indicate the empty space within the structure and the remaining substrate material. It may be determined either optically, i.e., from the analysis of microscope photographs, for example, or gravimetrically. In the case of the gravimetric determination, the following applies: Porosity P=(m1−m2)/(m1−m3), m1 being the mass of the sample before porosifying, m2 being the mass of the sample after porosifying, and m3 being the mass of the sample after etching using a 1-mole NaOH solution, which dissolves the porous structure chemically. Alternatively, the porous structure may also be dissolved by a KOH/isopropanol solution.

The advantage is that porosifying may be performed from the front of the Si-semiconductor substrate. In particular, porosifying does not have to be performed through the Si-semiconductor substrate or the Si wafer, nor do channels need to be provided for supplying the fluid through the Si-semiconductor substrate or the Si wafer, for example, via further trench steps.

The thickness of the porous layer may vary in a broad range as needed; only a thin surface layer may thus be porosified, or the porous layer may have a thickness of several 100 μm. The thickness of the porous layer may be in the range of ≧10 μm to ≦250 μm, and may be in the range of ≧20 μm to ≦150 μm, and may also be in the range of ≧50 μm to ≦100 μm. In certain exemplary embodiments, the semiconical microneedle may be completely porosified. One advantage of porosifying the semiconical microneedles is that the biocompatibility of the microneedles may be enhanced. Any debris remaining in the body may thus be broken down.

Porosified hollow needles and/or porosified semiconical microneedles without a through opening or a through channel through the inside of the microneedle structure may be manufactured.

The pore diameter is adjustable according to the hydrofluoric acid concentration, doping, and current density in a range from a few nanometers to a few μm. For example, pores having a diameter in the range of between ≧5 nm to ≦2 μm, which may be in the range of between ≧5 nm to ≦30 nm, may be manufactured.

The second masking layer is removed after the completion of the isotropic etching or porosification. For example, if nitride masks are used, they may be removed via further storage of the Si-semiconductor substrate in the electrolyte, during which the hydrofluoric acid-containing electrolyte etches away the masking layer.

It may be provided that the semiconical microneedles be used in the form of a contiguous system or an array. Suitable systems may be established by the appropriate selection of the masking layers. Optionally, the semiconical microneedles may be separated from the Si-semiconductor substrate, for example, in blocks, i.e., at least two semiconical microneedles, or the semiconical microneedles may be separated individually, i.e., as individual semiconical microneedles, and individual semiconical microneedles may be obtained for further use. The semiconical microneedles may be separated, for example, individually or in fields by cutting or sawing the semiconductor substrate. For example, the semiconical microneedles may be separated by sawing the Si-semiconductor substrate in areas or portions having a desired number of semiconical microneedles.

A further subject matter of the exemplary embodiments and/or exemplary methods of the present invention relates to semiconical microneedles which are manufacturable according to the method according to the present invention, the shaft of the semiconical microneedles including a vertical outer wall and a conical portion of the outer wall.

The vertical outer walls of the recesses produced form the vertical wall of a semiconical microneedle. The conical portion of the outer wall is formed in a subsequent method step by isotropic etching of the Si-semiconductor substrate.

A further subject matter of the exemplary embodiments and/or exemplary methods of the present invention relates to a device for releasing a substance into or through the skin, including at least one system of semiconical microneedles around at least one central recess, manufacturable according to the method according to the present invention. It is advantageous that the recesses between the semiconical microneedles may be used as reservoirs for the substances to be applied, for example, for drugs.

Basically, semiconical microneedles are suitable for any application requiring microneedles. In particular, the semiconical microneedles are suitable for biological applications, in particular for injecting substances such as drugs into or through the skin. In particular semiconical microneedles made of porous silicon are biocompatible and may be reabsorbed by the body. Semiconical microneedles made of porous silicon may also be used as reservoirs for the substances to be applied.

What is claimed is:

1. A method for manufacturing semiconical microneedles in a Si-semiconductor substrate, the method comprising:
a) applying and structuring a first masking layer on a outer surface of a front of an Si-semiconductor substrate, discrete holes having straight lateral edges and an average diameter in a range of ≧50 μm to ≦1000 μm being formed in the first masking layer;
b) producing recesses having vertical lateral walls in the Si-semiconductor substrate by anisotropic etching into the discrete holes of the first masking layer of the Si-semiconductor substrate, the lateral walls of the produced recesses forming a vertical wall of the semiconical microneedles;
c) removing the first masking layer;
d) applying and structuring a second masking layer on the outer surface of the front of the Si-semiconductor substrate, the recesses remaining masked and adjacent areas along the lateral edges of the recesses being masked, these areas being covered in a semicircular shape;
e) isotropically etching the front of the Si-semiconductor substrate, during which the conical wall of the semiconical microneedles is formed; and
g) removing the second masking layer;

2. The method of claim 1, wherein recesses having a square shape are formed.

3. The method of claim 1, wherein recesses are produced having at least one of: (i) a depth in the range of ≧100 μm to ≦500 μm, and (ii) an average diameter in the range of ≧50 μm to ≦200 μm.

4. The method of claim 1, wherein a channel is formed in the semiconical microneedles via isotropic etching of the Si-semiconductor substrate.

5. The method of claim 1, wherein the isotropic etching of the front of the Si-semiconductor substrate, during which a conical wall of a semiconical microneedle is formed, takes place by electrochemical anodizing.

6. The method of claim 1, wherein the isotropic etching of the front of the Si-semiconductor substrate, during which the conical wall of a semiconical microneedle is formed, is performed by a dry etching method using gases that etch silicon isotropically.

7. The method of claim 1, wherein the semiconical microneedles are porosified by electrochemical anodizing.

8. A semiconical microneedle comprising:
a Si-semiconductor substrate;
semiconical microneedles in the Si-semiconductor substrate, the microneeedles being made by performing the following:
a) applying and structuring a first masking layer on a outer surface of a front of an Si-semiconductor substrate, discrete holes having straight lateral edges and an average diameter in a range of ≧50 μm to ≦1000 μm being formed in the first masking layer;
b) producing recesses having vertical lateral walls in the Si-semiconductor substrate by anisotropic etching into the discrete holes of the first masking layer of the Si-semiconductor substrate, the lateral walls of the produced recesses forming a vertical wall of the semiconical microneedles;
c) removing the first masking layer;
d) applying and structuring a second masking layer on the outer surface of the front of the Si-semiconductor substrate, the recesses remaining masked and adjacent areas along the lateral edges of the recesses being masked, these areas being covered in a semicircular shape;
e) isotropically etching the front of the Si-semiconductor substrate, during which the conical wall of the semiconical microneedles is formed; and
g) removing the second masking layer;
wherein the shaft of the semiconical microneedle includes a vertical outer wall and a conical portion of the outer wall.

9. A device for releasing a substance into the skin, comprising:
at least one system of semiconical microneedles around at least one central recess, the microneeedles being made by performing the following:
a) applying and structuring a first masking layer on a outer surface of a front of an Si-semiconductor substrate, discrete holes having straight lateral edges and an average diameter in a range of ≧50 μm to ≦1000 μm being formed in the first masking layer;
b) producing recesses having vertical lateral walls in the Si-semiconductor substrate by anisotropic etching into the discrete holes of the first masking layer of the Si-semiconductor substrate, the lateral walls of the produced recesses forming a vertical wall of the semiconical microneedles;
c) removing the first masking layer;
d) applying and structuring a second masking layer on the outer surface of the front of the Si-semiconductor substrate, the recesses remaining masked and adjacent areas along the lateral edges of the recesses being masked, these areas being covered in a semicircular shape;
e) isotropically etching the front of the Si-semiconductor substrate, during which the conical wall of the semiconical microneedles is formed; and
g) removing the second masking layer.

10. A system of semiconical microneedles for the applying a substance through the skin, comprising:
microneeedles being made by performing the following:
a) applying and structuring a first masking layer on a outer surface of a front of an Si-semiconductor substrate, discrete holes having straight lateral edges and an average diameter in a range of ≧50 μm to ≦1000 μm being formed in the first masking layer;
b) producing recesses having vertical lateral walls in the Si-semiconductor substrate by anisotropic etching into the discrete holes of the first masking layer of the Si-semiconductor substrate, the lateral walls of the produced recesses forming a vertical wall of the semiconical microneedles;
c) removing the first masking layer;
d) applying and structuring a second masking layer on the outer surface of the front of the Si-semiconductor substrate, the recesses remaining masked and adjacent areas along the lateral edges of the recesses being masked, these areas being covered in a semicircular shape;
e) isotropically etching the front of the Si-semiconductor substrate, during which the conical wall of the semiconical microneedles is formed; and
g) removing the second masking layer.

11. The method of claim 1, the method further comprising at least one of:
f) porosifying the front of the Si-semiconductor substrate; and
h) separating the semiconical microneedles from the Si-semiconductor substrate.

12. The method of claim 1, wherein recesses are produced having at least one of: (i) a depth in the range of ≧150 μm to ≦250 μm, and (ii) an average diameter in the range of ≧100 μm to ≦150 μm.

13. The method of claim 1, wherein a channel is formed in the semiconical microneedles via isotropic etching of the Si-semiconductor substrate, and the channel is connected to the recess.

14. The method of claim 1, wherein the isotropic etching of the front of the Si-semiconductor substrate, during which a conical wall of a semiconical microneedle is formed, takes place by electrochemical anodizing, which includes a hydrofluoric acid-containing electrolyte.

15. The method of claim 1, wherein the isotropic etching of the front of the Si-semiconductor substrate, during which the conical wall of a semiconical microneedle is formed, is performed by a dry etching method using gases that etch silicon isotropically, which is selected from the group including SF₆, XeF₂ and ClF₃.

16. The method of claim 1, wherein the semiconical microneedles are porosified by electrochemical anodizing, which is in a hydrofluoric acid-containing electrolyte.
